(19) United States
(12) Patent Application Publication
(10) Pub. No.: US 2014/0182586 A1
(43) Pub. Date: Jul. 3, 2014

(54) Responders Support Unit

(71) Applicant: James Robert Glazier, Incline Village, NV (US)

(72) Inventor: James Robert Glazier, Incline Village, NV (US)

(21) Appl. No.: 13/554,088

(22) Filed: Jan. 2, 2013

(51) Int. Cl. A61N 1/39; A61M 16/10

(52) U.S. Cl. CPC A61N 1/3993; A61M 16/10
USPC 128/202.26

(57) ABSTRACT

This portable device, which allows people without medical experience, the ability to support persons in medical distress until more trained persons are available. With the use of audible instruction the user is guided in calling for medical assistance, placement of pulse oximeter and cardiac electrodes. The device continuously analyzes input from the pulse oximeter and cardiac electrodes. If input suggests that supplemental oxygen could benefit the person in distress, the user is guided by audible instruction in the use of the oxygen delivery system, and automatically activates the Chemical Oxygen Generator (COG) to start oxygen flow. If the input suggests that Cardiac Shock (Defibrillation) could benefit the person in distress, the audible instruction informs the user and directs the user accordingly, activating the Automated External Defibrillator (AFD). Audible instructions would be available in multiple languages as selected by the user.

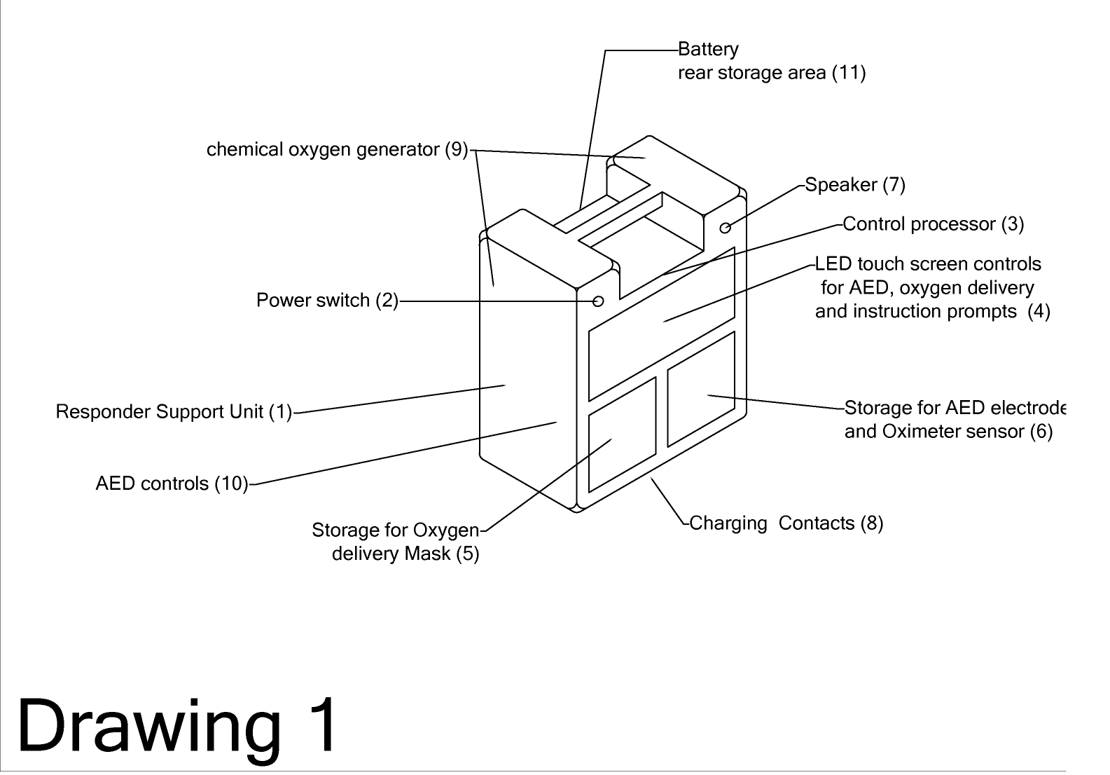

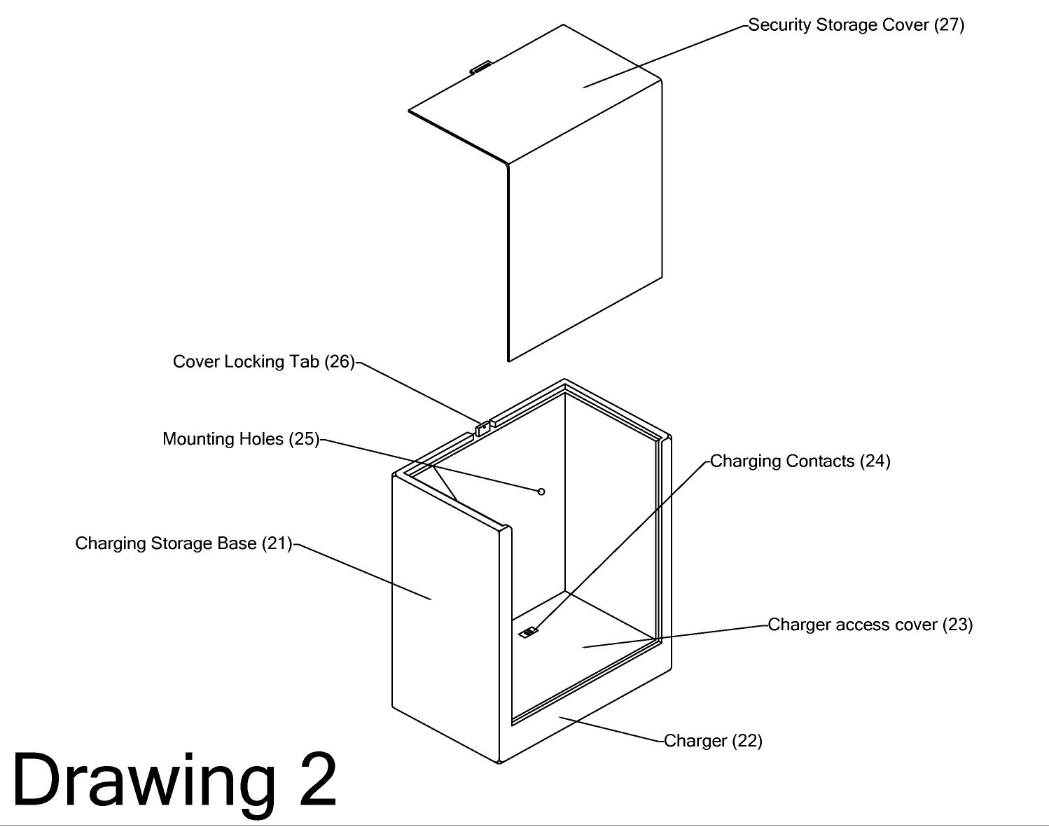

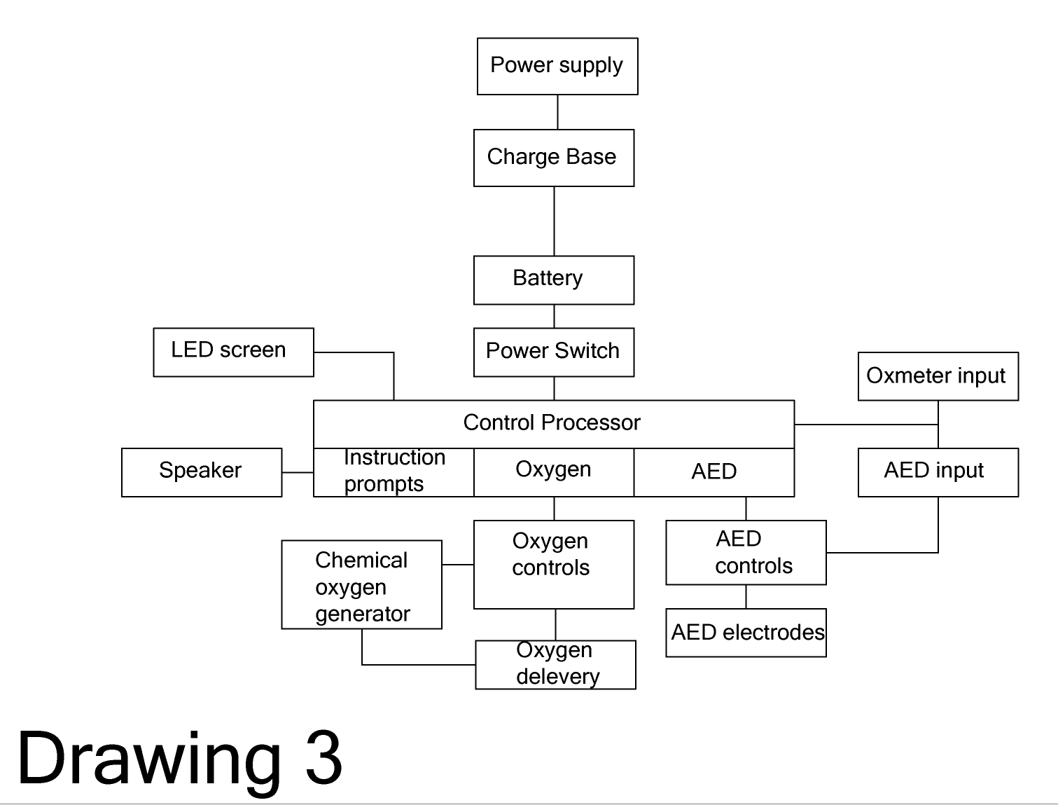

### INVENTION

This invention is designed to supplement the Emergency Medical System (EMS) by giving the general public a device which will allow them to start medical assistance until trained medical support is available.

### DESCRIPTION

Advances in emergency medicine, has over the years shown that persons (patients) in medical distress from poor oxygenation, causes injury to the brain. Insult to the brain due to poorly oxygenated blood caused damage in a very short periods of time. The quicker oxygenation levels are re-established to the brain, the better the outcome of the patient. Until underlying medical problems can be resolved, using supplemental oxygen to enrich the blood has had tremendous improvements in patient outcomes. Currently medical assistance has come from activating the Emergency Medical System (EMS). However, as office buildings and manufacturing facility's increase in size, cities have taller and taller buildings, people work or live in more remote areas and travel is done by air, response times of trained medical professionals increases. This delay in treatment also increases the potential in injury and possibly death for the patient.

Available devices for delivery of oxygen involve the use of oxygen stored under very high pressure in metal cylinders. This requires specialized training for its safe storage and use. Also, this storage system requires recurrent checking for pressure loss from the cylinder as well as periodic checks of the cylinder itself for integrity. This requires an ongoing maintenance program to assure the serviceability of the device when needed.

Medical research has also shown that decreased oxygenation to the brain can occur form decreased circulation of blood caused by injury to the heart. Supplemental oxygen can assist in this situation. However, some injuries to the heart can only to be resolved thru cardiac shock (defibrillation). The Automated External Defibrillator (AED) is the current device available for this purpose.

Portable devices combining oxygen delivery and an AED do exist, However, there use of highly compressed oxygen cylinders requires specialized training. This as well as the ongoing maintenance requirements makes them unsuitable for use in many situations.

### SUMMARY

The RESPONDERS SUPPORT UNIT (RSU) is an invention which gives the general public the ability to supply supplemental oxygen and cardiac shock (defibrillation), in an immediate manner, to a person in medical distress, from a portable device, that instructs them, is safe and simple to use and has a long storage life. A multi-lingual audible instruction system giving simple step by step direction guiding use. A bright LED screen gives visual guidance and a user input method. Color coded pulse oximeter and cardiac electrodes simplify application. The Chemical Oxygen Generator (COG) as a method to supply oxygen creates a simplicity and safety for general use. The chemical oxygen generator is a device releasing oxygen created by a chemical reaction. These would include but are not limited to oxygen releasing compounds such as inorganic superoxide, chlorate, perchlorate, and ozonides, or chemical gas separation such as a pressure swing adsorption. The Automated External Defibrillator (AED) adds increased ability to improve outcomes. Recharger base maintains battery charge while allowing rapid portable deployment.

With the battery charge maintained on the charging base, the components of this invention have been shown to last 10 years or more in a standby mode ready for use.

### TABLE OF DRAWINGS

Drawing 1 is a perspective view conceptualizing the configuration of the invention.

Drawing 2 is a perspective view conceptualizing the configuration of the storage/recharging base with depiction of the protection cover.

Drawing 3 is a flow diagram of the relationship of components of this invention

### DESCRIPTION OF DRAWINGS

1) Responders Support Unit housing is made of high impact plastic, with integrated molded handle allowing ease of carrying, while holding all components of the invention.

2) Switch to activate use of the invention

3) Control processor digital algorithm control all functions of the invention, including but not limited to, instruction prompts, chemical oxygen generator, AED system, and input devices (LED screen, pulse oximeter and electrodes).

4) LED touch screen for user input responses and user available controls.

5) Storage compartment for tubing and mask from COG supply system to victim in distress.

6) Storage compartment for pulse oximeter and AED electrodes from invention to victim in distress.

7) Speaker to allow audible prompts to the user.

8) Charging contacts match contacts in charging base to maintain battery charge in invention.

9) Compartments for the COG components.

10) AED components.

11) Battery

21) High impact plastic base for storage of the invention as well as supplying energy from an outside source for maintenance of the inventions battery charge.

22) Charger with cord from the outside energy supply to the charging contacts.

23) access cover to charger.

24) Charging contacts to connect to the invention.

25) Holes to mount storage base.

26) Locking tab to secure storage cover.

27) Clear acrylic housing cover to allow visibility of invention while stored awaiting use.

I claim the following:

1. The use of a Chemical Oxygen Generator (COG) as an oxygen delivery system in conjunction with an Automated External Defibrillator (AED) is an new invention.

2. The audible instruction algorithm guiding use of the an oxygen delivery system in conjunction with an Automated External Defibrillator (AED) by persons without medical experience is a new invention.
